SEQUENCE LISTING (2 sequences)

SEQ ID NO: 1 - protein, 14 aa, Crotalus durissus terrificus
EFSPENCQGESQPC

SEQ ID NO: 2 - protein, 14 aa, Artificial Sequence
  modified crotalphine peptide
EFSPENAQGESQPA

(19) United States
(12) Patent Application Publication
(10) Pub. No.: US 2020/0038474 A1
(43) Pub. Date: Feb. 6, 2020

(54) COMPOSITION, USE AND METHOD FOR TREATING NEURODEGENERATIVE DISEASES

(71) Applicant: FUNDAÇÃO BUTANTAN, SÃO PAULO (BR)

(72) Inventors: Gisele PICOLO, SÃO PAULO (BR); Yara CURY, SÃO PAULO (BR)

(21) Appl. No.: 16/375,060

(22) PCT Filed: Oct. 4, 2017
(86) PCT No.: PCT/BR2017/050304

§ 371 Date: Apr. 4, 2019

(30) Foreign Application Priority Data
Oct. 4, 2016 (BR) 10 2016 023153 1

(51) Int. Cl. A61K 38/10; A61P 25/28

(52) U.S. Cl. CPC A61K 38/10; A61P 25/28

(57) ABSTRACT

The present invention relates to a composition comprising SEQ ID NO:1 or 2 and one or more pharmaceutically acceptable carriers or diluents. The present invention further relates to the use of the composition as defined in claim 1 or SEQ ID NO:1 or 2 for the manufacture of a medicament for the treatment of neurodegenerative diseases. The present invention further relates to a method of treating neurodegenerative diseases.

This application incorporates by reference the contents of a 777 byte text file created on Oct. 17, 2019 and named “SN16375060substitutesequencelisting.txt,” which is the sequence listing for this application.

### TECHNICAL FIELD

The present invention is in the field of medicine. The present invention relates to a composition comprising SEQ ID NO: 1 or 2 and one or more pharmaceutically acceptable carriers or diluents. The present invention further relates to the use of the composition as defined in claim 1 or SEQ ID NO: 1 or 2 for the manufacture of a medicine for the treatment of neurodegenerative diseases. The present invention further relates to a method for treating neurodegenerative diseases.

### BACKGROUND OF THE INVENTION

Multiple sclerosis (MS) is a demyelinating chronic inflammatory disease of the central nervous system (CNS) that affects more than 2.5 million people worldwide. It is a disorder of autoimmune origin, where the immune system recognizes as antigens components of the CNS, more specifically, peptides that constitute the myelin sheath of axons of neurons (Finean, 1960). Several motor alterations, cognitive and sensory, which cause sensory loss, visual problems, muscle weakness, ataxia, fatigue, functional disorders of bladder and bowel, loss of memory, difficulties in coordination and speech, among others may be observed (Aicher et al, 2004; Slavin et al, 2010).

Pathologically, MS is characterized by demyelination plaques in the CNS white matter, accompanied by perivascular infiltrates of T cells, B cells and macrophages (Lucchinetti et al, 2000) and axonal damage in some patients (Lassmann et al, 1998). It is known that the pathogenesis of MS does not follow a single pathway, but from multiple signaling pathways that differ in specific triggers of the disease, the immune tolerance mechanisms and the type of effector cells that cause disease (Lucchinetti et al, 2000; Lassmann et al, 1998; Hemmer et al, 2002). It has been shown that axonal degeneration is the main cause of irreversible neurological changes in MS patients. This lesion is observed early in the disease and is correlated with the intensity of the inflammatory response (Bjartmar et al, 2003).

MS arises frequently in reproductive age (between 20 and 40 years) and as in many other autoimmune diseases, there is a greater incidence in women than in men, and several factors have been suggested to explain this difference, among them the female sex hormones, genetics and the environment (Tintoré & Arrambide, 2009; Eikelenboom et al, 2009). Also, it is important to emphasize that MS has no cure, and the therapy focuses on actions that delay the progression of the disease and improve the quality of life of the patient by promoting the relief of symptoms.

Patent Application 0502399-8, filed on May 2, 2005, in the name of Yara Cury, FAPESP & Biossintética Farmacêutica Ltda, entitled “ANALOGENIC COMPOUNDS TO ANALGESIC PEPTIDES DERIVED FROM CROTALUS DURISSUS TERRIFICUS SERPENTS VENOM, ITS USES, COMPOSITIONS, PURIFICATION AND PREPARATION METHODS”, describes similar compounds to peptides with amino acid sequences SEQ ID NO: 1, SEQ ID NO: 2, SEQ ID NO: 3 or SEQ ID NO: 4, including analgesic peptides derived from snakes of the species Crotalus durissus terrificus, their uses in the treatment, diagnosis and prevention of painful or mediated processes by opioid receptors, their compositions and their methods of preparation and purification, as well as their use in the identification of analgesic compounds. The above-mentioned patent application makes reference to the use of crotalphine only as analgesic.

The article “Crotalphine, a novel potent analgesic peptide from the venom of the South American rattlesnake Crotalus durissus terrificus”, published in Peptides, Vol. 29, pp. 1293-1304 (2008), by Konno, K; Picolo, G.; Gutierrez, V. P.; Brigatte, P.; Zambelli, V. O.; Camargo, A. C. M.; Cury, Y., describes that the venom of Crotalus durissus terrificus induces a long-term antinociceptive effect mediated by the activation of κ and δ opioid receptors. Although mediated by opioid receptors, prolonged treatment with crotalid venom would not cause the development of peripheral tolerance or withdrawal symptoms with its withdrawal. The results indicate that crotalphine induces antinociception mediated by κ opioid receptor activation and may contribute to the antinociceptive effect of crotalid venom. This article, therefore, also refers to the use of crotalphine only for the purpose of analgesia.

In the present application, studies with crotalphine were performed using the Experimental Autoimmune Encephalomyelitis model, a model that reproduces in animals many of the anatomical and behavioral changes observed in multiple sclerosis in humans. These models have been used to study both the development and progression of the disease, and for pre-clinical trials to evaluate new medicines with therapeutic potential (Sloane et al, 2009) and reproduce inflammation in the CNS demyelination of neurons and motor alterations observed in MS (Baxter, 2007; Olechowski et al, 2009; Basso et al, 2008). In the present application, the model of Experimental Autoimmune Encephalomyelitis induced by MOG35-55 was used.

The present application demonstrates the efficacy of crotalphine in an animal model of multiple sclerosis, demonstrating that crotalphine is able to partially reverse the motor impairment observed in this disease. Multiple sclerosis is a disease that has no cure, and therapy focuses on actions that delay the progression of the disease and improve the quality of life of the patient by promoting relief of symptoms. Thus, the data of the present application suggest the therapeutic potential of crotalphine in neurodegenerative diseases.

### SUMMARY OF THE INVENTION

The above-mentioned article and patent application describe the use of crotalphine only for analgesic purposes, and there is no suggestion in the prior art of using crotalphine for the manufacture of medicines in order to treat neurodegenerative diseases such as multiple sclerosis. Therefore, the present invention proposes a completely different use for crotalphine from that disclosed in the prior art, thus disclosing a compound to be used, alternatively, for the therapy of such diseases.

In one aspect, the present invention relates to a composition comprising SEQ ID NO: 1 or 2 and one or more pharmaceutically acceptable carriers or diluents.

In another aspect, the present invention relates to the use of the composition as defined in claim 1 or the sequence as defined in SEQ ID NO: 1 or 2 for the manufacture of a medicament for the treatment of neurodegenerative diseases. In one embodiment, the neurodegenerative disease is selected from Alzheimer's disease, Parkinson's disease, Huntington's Disease, Multiple Sclerosis, Amyotrophic Lateral Sclerosis, autoimmune diseases, hepatitis, and inflammatory and degenerative rheumatism including rheumatoid arthritis. In a further embodiment, the neurodegenerative disease is Multiple Sclerosis.

In another aspect, the present invention relates to a method of treating neurodegenerative diseases by administering the composition as defined in claim 1 or the sequence as defined in SEQ ID NO: 1 or 2 to a patient suffering from said disease. In one embodiment, the neurodegenerative disease is selected from Alzheimer's disease, Parkinson's disease, Huntington's disease, Multiple Sclerosis, Amyotrophic Lateral Sclerosis, autoimmune diseases, hepatitis, and inflammatory and degenerative rheumatism including rheumatoid arthritis. In a further embodiment, the neurodegenerative disease is Multiple Sclerosis.

### BRIEF DESCRIPTION OF THE DRAWINGS

The objective of the invention, together with additional advantages thereof, may be better understood by reference to the accompanying figures and the following descriptions:

FIG. 1A shows the effect of crotalphine on clinical signs of animals with experimental autoimmune encephalomyelitis. Data represent mean±SEM (n=8). *p<0.05 compared to the control group (two-way ANOVA, Bonferroni as post hoc test).

FIG. 1B shows the effect of crotalphine on clinical signs of animals with experimental autoimmune encephalomyelitis. The area of the graph under the curve is expressed as mean±SEM (n=8). *p<0.05 compared to the control group (two-way ANOVA, Bonferroni post hoc test).

### DETAILED DESCRIPTION OF THE INVENTION

Although the present invention may be susceptible to different embodiments, it is shown in the drawings and in the following detailed discussion, a preferred embodiment with the understanding that the present disclosure is to be considered an exemplification of the principles of the invention and is not intended to limit the present invention to which was illustrated and described herein.

Composition

In a first embodiment, the present invention relates to a composition comprising SEQ ID NO: 1 or 2 and one or more pharmaceutically acceptable carriers or diluents.

SEQ ID NO: 1 refers to the peptide sequence termed crotalphine, namely, EFSPENCQGESQPC.

SEQ ID NO: 2 refers to the modified crotalphine peptide sequence, namely, EFSPENAQGESQPA.

Examples of pharmaceutical forms, carriers, diluents and pathways of administration comprised by the present invention are described (but not limited to) in Remington's Pharmaceutical Sciences, 17th ed., Mack Publishing Company, Easton, Pa., USA. Carriers or diluents as used in the present invent/ion relate to a non-toxic, inert solid, semi-solid liquid excipient, diluent, auxiliary formulation of any type, such as saline and water. Some examples of the materials that may serve as pharmaceutically acceptable carriers are sugars, such as lactose, glucose and sucrose, starches such as corn starch and potato starch, cellulose and its derivatives, such as sodium carboxymethylcellulose, ethylcellulose and cellulose acetate, cyclodextrin; oils such as peanut oil, cottonseed oil, sunflower oil, sesame oil, olive oil, corn oil and soybean oil; glycols, such as propylene glycol, polyols, such as glyceringlycol, sorbitol, mannitol and polyethylene; esters, such as ethyl laurate, ethyl oleate, agar; buffering agents, such as aluminum hydroxide and magnesium hydroxide; alginic acid; isotonic saline, Ringer's solution; buffer solutions of ethyl alcohol and phosphate, oily emulsion in water containing mycobacteria killed by the action of heat or components of its cell wall (complete Freund's adjuvant), as well as other compatible non-toxic substances used in pharmaceutical formulations.

### Use

In a second embodiment, the present invention relates to the use of the composition as defined in claim 1 or the sequence as defined in SEQ ID NO: 1 or 2 for the manufacture of a medicament for the treatment of neurodegenerative diseases. In a further embodiment, the neurodegenerative disease is selected from Alzheimer's disease, Parkinson's disease, Huntington's Disease, Multiple Sclerosis, Amyotrophic Lateral Sclerosis, autoimmune diseases, hepatitis, and inflammatory and degenerative rheumatism including rheumatoid arthritis. In yet another embodiment, the neurodegenerative disease is Multiple Sclerosis.

### Method for Treating Neurodegenerative Diseases

In a third embodiment, the present invention relates to a method for treating neurodegenerative diseases by administering the composition as defined in claim 1 or the sequence as defined in SEQ ID NO: 1 or 2 to a patient suffering from said disease. In one embodiment, the neurodegenerative disease is selected from Alzheimer's disease, Parkinson's disease, Huntington's Disease, Multiple Sclerosis, Amyotrophic Lateral Sclerosis, autoimmune diseases, hepatitis, and inflammatory and degenerative rheumatism including rheumatoid arthritis. In a further embodiment, the neurodegenerative disease is Multiple Sclerosis.

### EXAMPLE

Experimental Autoimmune Encephalomyelitis (EAE) was induced by the immunization of female C57BL/6 mice with MOG35-55 (200 μg) and Mycobacterium tuberculosis (400 μg) in incomplete Freund's adjuvant, followed by injection of pertussis toxin (300 ng, on days 0 and 2) (Adapted from Basso A. S. et al., 2008). The clinical signs were evaluated according to scores from 0 to 5 [0, absence of symptoms; 1, loss of tail tonus; 2, partial paralysis of the hind limbs; 3, total paralysis of hind limbs; 4, total paralysis of the hind limbs and partial paralysis of the anterior ones; 5, decreased responsiveness and death (euthanasia). Animals that receive grade 4 are evaluated 2 times a day and in the occurrence of 3 consecutive grades 4, they receive euthanasia.

The results obtained in our studies show that the clinical signs appear on the 10th day in animals with EAE, with a peak on the 18th day, which is maintained throughout the evaluation period.

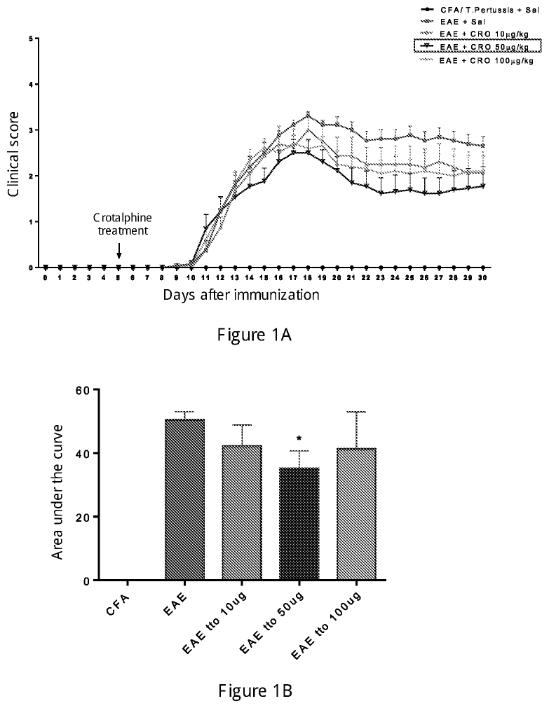

Crotalphine, SEQ ID NO: 1, administered as a single dose on the 5th day after immunization at 50 μg/kg dose significantly decreased the severity of clinical signs, where 43% of the animals reached clinical score 2, while 100% of the animals in the group control, treated with saline, reached a score between 3 and 5 (FIGS. 1A and B).

### REFERENCES

Aicher, S. A.; Silverman, M. B.; Winkler, C. W.; Bebo, B. F., Pain, 110, p. 560-570, 2004.

Basso, A. S.; Frenkel, D.; Quintana, F. J.; Costa-Pinto, F. A.; Petrovic-Stojkovic, S.; Puckett, L.; Monsonego, A.; Bar-Shir, A.; Engel, Y.; Gozin, M.; Weiner, H. L. J Clin Invest, 118, p. 1532-1543, 2008.

Baxter, A. G. Nat Rev Immunol, 7, 904-912, 2007.

Bjartmar C.; Wujek, J. R.; Trapp, B. D. Journal of the Neurological Sciences, 206, p. 165-171, 2003.

Eikelenboom, M. J.; Killestein, J.; Kragt, J. J.; Uitdehaag, B. M. J.; Polman, C. H., Journal of the Neurological Sciences, 286, p. 40-42, 2009.

Finean, J. B. J Biophys Biochem Cytol 8, p. 13-29, 1960.

Hemmer, B.; Cepok, S.; Nessler, S.; Sommer, N. Curr Opin Neurol, 15, p. 227-231, 2002.

Lassmann, H.; Raine, C. S.; Antel, J.; Prineas, J. W.; J Neuroimmunol, 86, p. 213-217, 1998.

Lucchinetti C.; Bruck, W.; Parisi, J.; Scheithauer, B.; Rodriguez, M; Lassmann, H., Ann Neurol, 47, p. 707-717, 2000.

Olechowski, C. J.; Parmar, A.; Miller, B.; Stephan, J.; Tenorio G.; Tran, K.; Leighton, J.; Kerr, B. J. Pain, 149, p. 565-572, 2009.

Slavin, A.; Kelly-Modis, L.; Labadia, M.; Ryan, K.; Brown, M. L., Autoimmunity, 43, p. 504-513, 2010.

Sloane, E.; Ledeboer, A.; Seibert, W.; Coats, B.; van Strien, M.; Maier, S. F.; Johnson, K. W.; Chavez, R.; Watkins, L. R.; Leinwand, L.; Milligan, E. D.; Van Dam, A. M. Brain, Behavior, and Immunity, 23, p. 92-100, 2009.

Tintoré, M. & Arrambide, G. Journal of the Neurological Sciences, 286, p. 31-34, 2009.

1. A composition, characterized in that it comprises SEQ ID NO: 1 or 2 and one or more pharmaceutically acceptable carriers or diluents.

2-4. (canceled)

5. A method for treating neurodegenerative diseases characterized in that the composition as defined in claim 1 or the sequence as defined in SEQ ID NO: 1 or 2 is administrated to a patient suffering from said disease.

6. The method according to claim 5, wherein the neurodegenerative disease is selected from the group consisting of Alzheimer's disease, Parkinson's disease, Huntington's Disease, Multiple Sclerosis, Amyotrophic Lateral Sclerosis, autoimmune diseases, hepatitis, and inflammatory and degenerative rheumatism.

7. The method according to claim 5, wherein the neurodegenerative disease is multiple sclerosis.

8. The method of claim 6, wherein the neurodegenerative disease is degenerative rheumatism and wherein the degenerative rheumatism is rheumatoid arthritis.
